(19) United States
(12) Patent Application Publication
(10) Pub. No.: US 2026/0294215 A1
(43) Pub. Date: Oct. 1, 2026

(54) ATTACHMENT FOR MEDICAL INSTRUMENT AND MEDICAL INSTRUMENT SYSTEM

(71) Applicant: FUJIFILM Corporation, Tokyo (JP)

(72) Inventors: Kohei HIGASHI, Kanagawa (JP); Keno KANAI, Kanagawa (JP)

(73) Assignee: FUJIFILM Corporation, Tokyo (JP)

(21) Appl. No.: 19/630,372

(22) Filed: Mar. 26, 2026

(30) Foreign Application Priority Data
Mar. 28, 2025 (JP) 2025-055198

(51) Int. Cl. A61B 1/00; A61B 1/005

(52) U.S. Cl. CPC A61B 1/00105; A61B 1/00042; A61B 1/0052

(57) ABSTRACT

An attachment for a medical instrument is easily attachable to and detachable from an operating member of a medical instrument. A bending operation knob attachment (attachment for a medical instrument) includes a top surface portion provided at a position facing an end surface of a bending operation knob on one side in a rotation axis A direction, and a side wall portion extending from an edge portion of the top surface portion toward the other side opposite to the one side in the rotation axis A direction. The side wall portion includes a first form portion provided at a position facing a recessed surface portion of the bending operation knob, and the first form portion includes a first protruding portion provided on a facing surface that faces the recessed surface portion and protruding toward the recessed surface portion to come into contact with the recessed surface portion.

### CROSS-REFERENCE TO RELATED APPLICATION

The present application claims priority under 35 U.S.C §119(a) to Japanese Patent Application No. 2025-055198 filed on Mar. 28, 2025, which is hereby expressly incorporated by reference, in its entirety, into the present application.

### BACKGROUND OF THE INVENTION

### Field of the Invention

The present invention relates to an attachment for a medical instrument, the attachment being attachable to an operating member of a medical instrument and a medical instrument system.

### Description of the Related Art

An endoscope, which is one of medical instruments, performs a bending operation on a bendable portion of an insertion portion by operating a bending operation knob provided in an operating portion of the endoscope. Accordingly, a distal end portion of the insertion portion can be oriented in a desired direction.

JP3181928Y discloses a bending operation knob attachment that is attachable to and detachable from a bending operation knob. The bending operation knob attachment includes a peripheral wall portion that covers an outer peripheral surface of the bending operation knob and a side wall portion that covers an outer side end surface near the outer peripheral surface.

The peripheral wall portion includes a first protruding portion that is inclined inward toward an attachment-side end surface of the bending operation knob, and the bending operation knob includes a first inclined portion that is inclined inward from a bulging portion of the finger hook recess toward the attachment-side end surface. The bending operation knob attachment is attached to the bending operation knob by bringing the first protruding portion into close contact with the first inclined portion.

### SUMMARY OF THE INVENTION

However, since the bending operation knob attachment (attachment for a medical instrument) disclosed in JP3181928Y is configured to attach to the bending operation knob (operating member) by bringing the first protruding portion and the first inclined portion that are inclined with respect to each other into close contact with each other, it may be difficult to attach to and detach from the bending operation knob due to variations in dimensional accuracy of the first protruding portion or the first inclined portion.

The present invention has been made in view of such circumstances, and an object thereof is to provide an attachment for a medical instrument that is easily attachable to and detachable from an operating member of a medical instrument and a medical instrument system.

An attachment for a medical instrument according to a first aspect of the present invention, the attachment being attachable to and detachable from an operating member rotatably provided in an operating portion of the medical instrument, in which a recessed surface portion for hooking a finger, which is recessed inward in a radial direction orthogonal to a rotation axis direction of the operating member, is provided in an outer peripheral portion of the operating member, includes: a top surface portion provided at a position facing an end surface of the operating member on one side in the rotation axis direction; and a side wall portion extending from an edge portion of the top surface portion toward the other side opposite to the one side in the rotation axis direction, in which the side wall portion includes a first form portion provided at a position facing the recessed surface portion, and the first form portion includes a first protruding portion provided on a first facing surface that faces the recessed surface portion and protruding toward the recessed surface portion.

In the attachment for a medical instrument according to a second aspect of the present invention, according to the first aspect, a plurality of the recessed surface portions are provided in the outer peripheral portion of the operating member, and the first protruding portion is provided at a position corresponding to only a part of the plurality of recessed surface portions.

In the attachment for a medical instrument according a third aspect of the present invention, according to the first aspect, a plurality of the recessed surface portions are provided in the outer peripheral portion of the operating member along a circumferential direction centered on the rotation axis direction, and the first protruding portion is provided at a position corresponding to every other recessed surface portion among the plurality of recessed surface portions along the circumferential direction.

In the attachment for a medical instrument according to a fourth aspect of the present invention, according to the first aspect, a protruding surface portion disposed on an outer side of the recessed surface portion in the radial direction is provided in the outer peripheral portion of the operating member, the side wall portion includes a second form portion provided at a position facing the protruding surface portion, and the top surface portion includes a second protruding portion disposed in a region of a second facing surface that faces the end surface on an inner side of the second form portion in the radial direction and protruding toward the end surface.

In the attachment for a medical instrument according to a fifth aspect of the present invention, according to the fourth aspect, a plurality of the recessed surface portions and a plurality of the protruding surface portions are alternately provided in the outer peripheral portion of the operating member along a circumferential direction centered on the rotation axis direction, the first protruding portion is provided at a position corresponding to every other recessed surface portion among the plurality of recessed surface portions along the circumferential direction, and the second protruding portion is provided at a position corresponding to every other protruding surface portion among the plurality of protruding surface portions along the circumferential direction.

In the attachment for a medical instrument according to a sixth aspect of the present invention, according to any one of the first aspect to the fifth aspect, the first protruding portion is provided to extend in the rotation axis direction.

In the attachment for a medical instrument according to a seventh aspect of the present invention, according to the fourth aspect or the fifth aspect, the second form portion includes a protrusion provided on an outer side surface facing outward in the radial direction.

In the attachment for a medical instrument according to an eighth aspect of the present invention, according to any one of the first aspect to the seventh aspect, the attachment for a medical instrument has an appearance with a color different from a color of the operating member.

In the attachment for a medical instrument according to a ninth aspect of the present invention, according to any one of the first aspect to the eighth aspect, a structural body including the top surface portion and the side wall portion is configured as at least a double-layer structure including an exterior member and an interior member surrounded by the exterior member and defining a space portion that accommodates the operating member.

In the attachment for a medical instrument according to a tenth aspect of the present invention, according to the ninth aspect, the interior member is made of a material having a lower hardness than a hardness of the exterior member.

In the attachment for a medical instrument according to an eleventh aspect of the present invention, according to the tenth aspect, the exterior member is a rigid member, and the interior member is an elastic member.

In the attachment for a medical instrument according to a twelfth aspect of the present invention, according to any one of the ninth aspect to the eleventh aspect, the structural body is a two-color molded product in which the exterior member and the interior member are integrated.

In the attachment for a medical instrument according to a thirteenth aspect of the present invention, according to any one of the ninth aspect to the twelfth aspect, the structural body includes an opening portion penetrating the top surface portion, and an edge portion of the opening portion is made of the interior member.

In the attachment for a medical instrument according to a fourteenth aspect of the present invention, according to any one of the first aspect to the thirteenth aspect, the medical instrument is an endoscope, and the operating member is a bending operation member receiving a bending operation on an insertion portion of the endoscope.

A medical instrument system according to a fifteenth aspect of the present invention includes: the medical instrument; and the attachment for a medical instrument according to any one of the first aspect to the fourteenth aspect.

In the medical instrument system according to a sixteenth aspect of the present invention, the medical instrument is an endoscope, and the operating member is a bending operation member receiving a bending operation on an insertion portion of the endoscope.

According to the present invention, the attachment for a medical instrument is easily attachable to and detachable from the operating member of the medical instrument.

### BRIEF DESCRIPTION OF THE DRAWINGS

FIG. 1 is an overall view of an endoscope to which a bending operation knob attachment according to an embodiment is attached.

FIG. 2 is an enlarged perspective view showing a configuration of an operating portion of the endoscope shown in FIG. 1.

FIG. 3 is a cross-sectional view taken along line III-III of the operating portion shown in FIG. 1.

FIG. 4 is a front view of the bending operation knob attachment.

FIG. 5 is a rear view of the bending operation knob attachment.

FIG. 6 is a perspective view of the bending operation knob attachment.

FIG. 7 is a perspective view of the bending operation knob attachment.

FIG. 8 is a side view of the bending operation knob attachment.

FIG. 9 is a main part enlarged perspective view showing a state in which the bending operation knob attachment is attached to the bending operation knob.

FIG. 10 is a rear view of a bending operation knob attachment according to a first modification example.

FIG. 11 is a rear view of a bending operation knob attachment according to a second modification example.

FIG. 12 is a rear view of a bending operation knob attachment according to a third modification example.

### DESCRIPTION OF THE PREFERRED EMBODIMENTS

Hereinafter, an embodiment of an attachment for a medical instrument and a medical instrument system according to the present invention will be described with reference to the accompanying drawings.

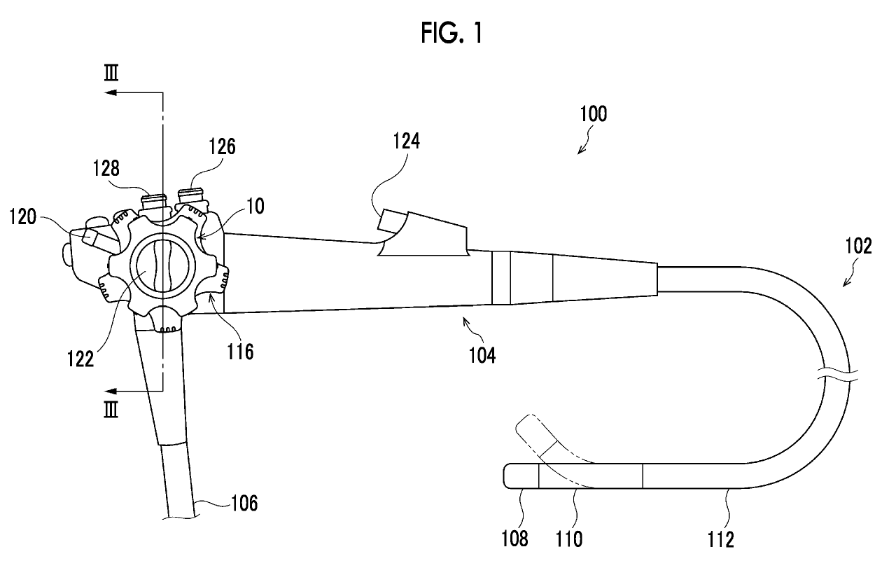

FIG. 1 is an overall view of an endoscope system including a bending operation knob attachment 10 according to the embodiment and an endoscope 100. The bending operation knob attachment 10 is an example of an attachment for a medical instrument according to the embodiment of the present invention. The endoscope 100 is an example of a medical instrument and an endoscope according to the present invention. The endoscope system is an example of a medical instrument system according to the embodiment of the present invention.

### Endoscope 100

As shown in FIG. 1, the endoscope 100 includes an insertion portion 102 that is inserted into a body, an operating portion 104 that is connected to a base portion of the insertion portion 102, and a universal cord 106 that is connected to the operating portion 104 and is connected to a processor device (not shown) that is an external device.

The insertion portion 102 includes a distal end portion 108, a bendable portion 110, and a soft portion 112 in order from the distal end.

An observation window (not shown) is provided in the distal end portion 108. An image of an observation site incident from the observation window is imaged by an imaging unit. An image signal obtained by the imaging unit is transmitted to the processor device via a signal cable inserted into the insertion portion 102, the operating portion 104, and the universal cord 106. Various types of image processing are performed on the image signal by the processor device, and the image is displayed on a monitor as an observation image.

In addition, an illumination window (not shown) is provided in the distal end portion 108. Illumination light is emitted from the illumination window toward the observation site. A light guide that transmits the illumination light is inserted into the insertion portion 102, the operating portion 104, and the universal cord 106 and is connected to a light source device via a connector. In addition, a forceps outlet (not shown) is provided in the distal end portion 108. A treatment tool that treats a diseased part is led out to the outside from the forceps outlet. In addition, an air/water supply nozzle is provided in the distal end portion 108. A liquid such as water or air is jetted from the air/water supply nozzle toward the observation site.

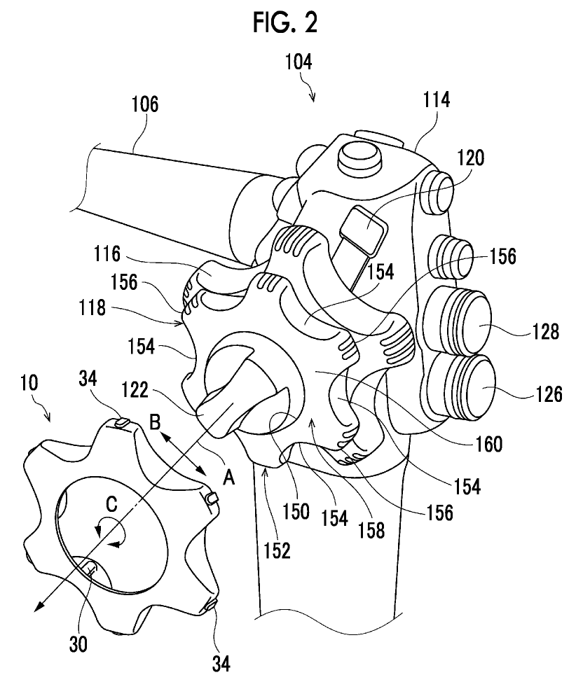

The bendable portion 110 is connected to the base portion of the distal end portion 108 and performs a bending operation in an up-down direction and a left-right direction in conjunction with a rotation operation of two bending operation knobs 116, 118 (see FIG. 2) described below.

The soft portion 112 has flexibility and connects the operating portion 104 and the bendable portion 110. The soft portion 112 has a predetermined length (for example, several meters) in order to reach the distal end portion 108 to a desired position in the body.

FIG. 2 is a perspective view showing a configuration of the operating portion 104. FIG. 2 shows a state in which the bending operation knob attachment 10 according to the embodiment is detached from the bending operation knob 118 of the operating portion 104 to a side of the operating portion 104.

As shown in FIG. 2, the operating portion 104 includes a case 114, the bending operation knob 116 for up-down, the bending operation knob 118 for left-right, a lock lever 120, and a lock dial 122. The operating portion 104 is an example of an operating portion of a medical instrument.

The case 114 is an exterior member that constitutes an outer surface of the operating portion 104. The bending operation knob 116 is an operating member that bends the bendable portion 110 in the up-down direction. The bending operation knob 118 is an operating member that bends the bendable portion 110 in the left-right direction. The lock lever 120 is an operating member that switches between locking and unlocking the rotation operation of the bending operation knob 116. The lock dial 122 is an operating member that switches between locking and unlocking the rotation operation of the bending operation knob 118. The up-down direction and the left-right direction correspond to, for example, an up-down direction and a left-right direction of an observation image displayed on a monitor.

The operating portion 104 includes a forceps port 124 (see FIG. 1), an air/water supply button 126, and a suction button 128 in addition to each of the above-described operating members.

A treatment tool such as a forceps or an injection needle used for treating a diseased part is inserted into the forceps port 124. The treatment tool is led out to the outside from the forceps outlet provided in the distal end portion 108 via a forceps channel inserted into the insertion portion 102.

The air/water supply button 126 is operated to supply a liquid such as water or air to an air/water supply channel inserted into the insertion portion 102. The liquid or air supplied to the air/water supply channel is jetted from the air/water supply nozzle described above toward the observation site.

The suction button 128 is operated to suction a suction target such as a liquid or tissue in the body from the forceps outlet via the forceps channel.

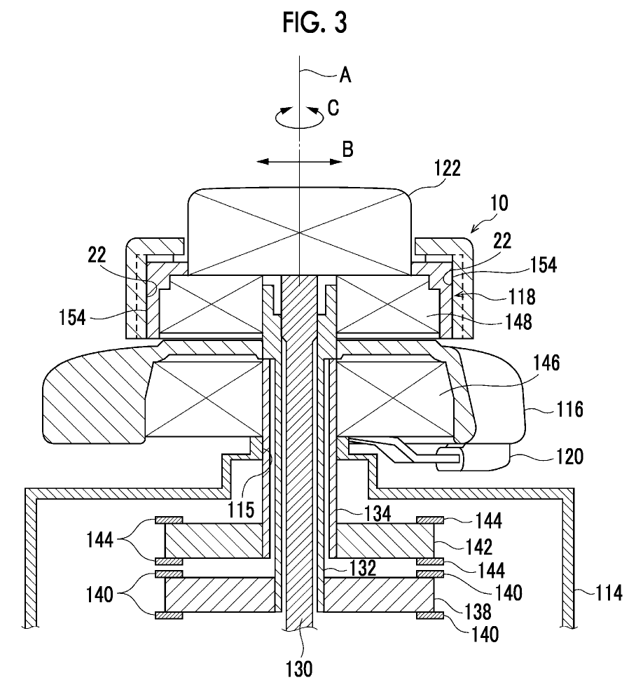

FIG. 3 is a cross-sectional view taken along line III-III of the operating portion 104 shown in FIG. 1. As shown in FIG. 3, the case 114 includes a support shaft 130.

The support shaft 130 is fixed to the case 114 and supports the entire bending operation mechanism of the operating portion 104. The support shaft 130 protrudes to the outer side (outside) of the operating portion 104 through an opening portion 115 formed in the case 114. The lock lever 120, the bending operation knob 116, the bending operation knob 118, and the lock dial 122 are attached to the protruding support shaft 130 in order from the bottom of FIG. 3.

A rotation drive cylinder 132 for left-right and a rotation drive cylinder 134 for up-down are disposed on an outer periphery of the support shaft 130. The rotation drive cylinder 132 is connected to the bending operation knob 118 on the outer side of the case 114 and rotates around the support shaft 130 in conjunction with the rotation operation of the bending operation knob 118. The rotation drive cylinder 134 is connected to the bending operation knob 116 on the outer side of the case 114 and rotates around the support shaft 130 in conjunction with the rotation operation of the bending operation knob 116.

The rotation drive cylinder 132 is connected to a sprocket 138 on an inner side (inside) of the case 114, a chain 140 is mounted on the sprocket 138, and base ends of left-right operation wires (not shown) are connected to both ends of the chain 140, respectively. Each operation wire is inserted into the soft portion 112, and a distal end thereof is connected to the bendable portion 110. As a result, in a case where the bending operation knob 118 is rotationally operated, the sprocket 138 rotates in response to the rotation direction, each operation wire is pulled, and the bendable portion 110 is bent in the left-right direction.

The rotation drive cylinder 134 is connected to a sprocket 142 on an inner side of the case 114, a chain 144 is mounted on the sprocket 142, and base ends of up-down operation wires (not shown) are connected to both ends of the chain 144, respectively. Each operation wire is inserted into the soft portion 112, and a distal end thereof is connected to the bendable portion 110. As a result, in a case where the bending operation knob 116 is rotationally operated, the sprocket 142 rotates in response to the rotation direction, each operation wire is pulled, and the bendable portion 110 is bent in the up-down direction.

The bending operation knob 116 includes an up-down locking mechanism 146 that is locked and unlocked in conjunction with the rotation operation of the lock lever 120 inside the bending operation knob 116. In a case where the lock lever 120 is moved to a predetermined lock position, the locking mechanism 146 locks the rotation operation of the bending operation knob 116. As a result, the bending state of the bendable portion 110 in the up-down direction is maintained. On the other hand, in a case where the lock lever 120 is moved to a predetermined unlock position, the locking of the bending operation knob 116 is released, and the bending operation of the bendable portion 110 in the up-down direction is possible. The configuration of the locking mechanism 146 is well known, and thus the description thereof will be omitted.

The bending operation knob 118 includes a left-right locking mechanism 148 that is locked and unlocked in conjunction with the rotation operation of the lock dial 122 inside the bending operation knob 118. In a case where the lock dial 122 is moved to a predetermined lock position, the locking mechanism 148 locks the rotation operation of the bending operation knob 118. As a result, the bending state of the bendable portion 110 in the left-right direction is maintained. On the other hand, in a case where the lock dial 122 is moved to a predetermined unlock position, the locking of the bending operation knob 118 is released, and the bending operation of the bendable portion 110 in the left-right direction is possible. The locking mechanism 148 also has the same configuration as the locking mechanism 146.

### Bending operation knob 118

Next, the bending operation knob 118 that is a target for attachment of the bending operation knob attachment 10 according to the embodiment will be described. The bending operation knob 118 is an example of an operating member of a medical instrument according to the present invention. In addition, the bending operation knob 118 is also an example of a bending operation member that receives a bending operation on the insertion portion of the endoscope.

As shown in FIG. 2, the bending operation knob 118 is rotatably provided in the operating portion 104 of the endoscope 100. That is, the bending operation knob 118 is rotated around a rotation axis A that is a central axis of the rotation drive cylinder 132 (see FIG. 3). In addition, the bending operation knob 118 includes an opening portion 150 at a central portion thereof, and the lock dial 122 protrudes to the outer side from the opening portion 150, so that the lock dial 122 can be operated by the operator.

In addition, the bending operation knob 118 includes an outer peripheral portion 152 that faces an outer side in a radial direction B orthogonal to the rotation axis A direction of the bending operation knob 118. A recessed surface portion 154 for hooking a finger of the operator is provided in the outer peripheral portion 152. The recessed surface portion 154 has an arc-shaped surface recessed toward the inner side in the radial direction B.

A plurality (six in the present example) of the recessed surface portions 154 are provided in the outer peripheral portion 152 along a circumferential direction C centered on the rotation axis A direction. In addition, the recessed surface portion 154 is a surface having a width in the rotation axis A direction (thickness direction of the bending operation knob 118). In the present example, the bending operation knob 118 having six recessed surface portions 154 is illustrated, but the present invention is not limited thereto. The number of the recessed surface portions 154 may be seven or more, may be five or less, or may be at least one. The recessed surface portion 154 is an example of a recessed surface portion for hooking a finger.

A protruding surface portion 156 is provided between a pair of recessed surface portions 154 adjacent to each other in the circumferential direction C in the outer peripheral portion 152. The protruding surface portion 156 has a shape that protrudes relatively outward in the radial direction B with respect to the recessed surface portion 154. A plurality (six in the present example) of the protruding surface portions 156 configured as described above are provided in the outer peripheral portion 152 along the circumferential direction C. That is, six recessed surface portions 154 and six protruding surface portions 156 are alternately provided in the outer peripheral portion 152 of the bending operation knob 118 along the circumferential direction C. The protruding surface portion 156 is an example of a protruding surface portion provided in the outer peripheral portion of the operating member.

A wall portion 158 that faces one side (side opposite to a side on which the case 114 is disposed) in the rotation axis A direction of the bending operation knob 118 is provided in the bending operation knob 118. The opening portion 150 is formed at a central portion of the wall portion 158. In addition, an end surface 160 that is a surface facing one side in the rotation axis A direction of the bending operation knob 118 is formed in the wall portion 158. The end surface 160 is an example of an end surface formed on one side of the operating member in the rotation axis direction.

### Bending operation knob attachment 10

Next, the bending operation knob attachment 10 according to the embodiment will be described. The bending operation knob attachment 10 is attachable to and detachable from the bending operation knob 118. Hereinafter, the bending operation knob attachment 10 will be described as an attachment 10.

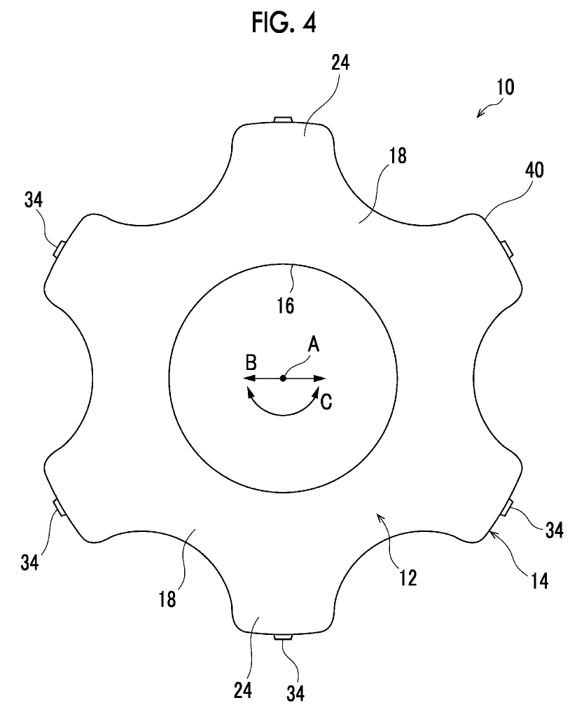

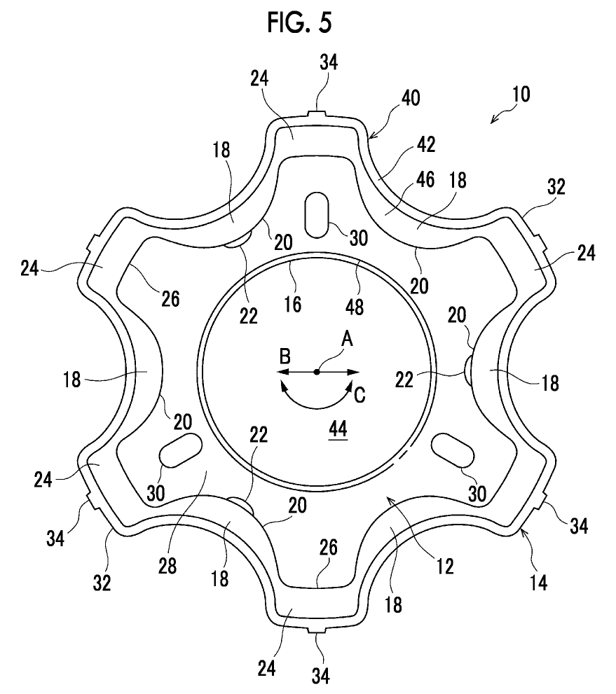

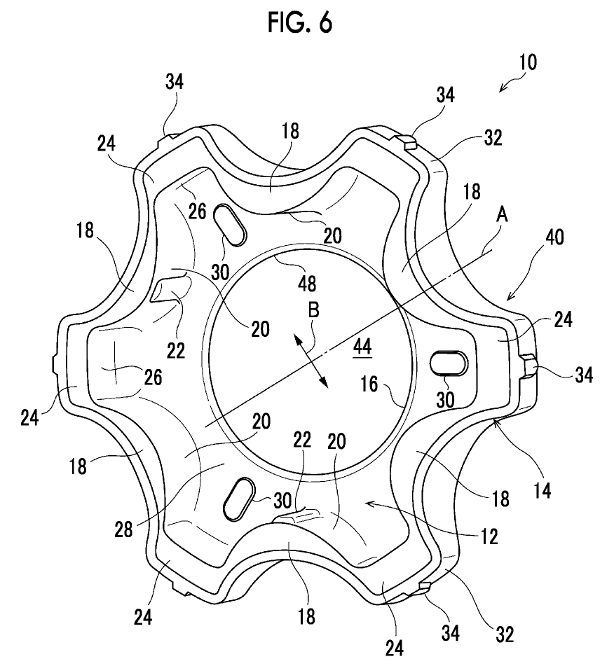

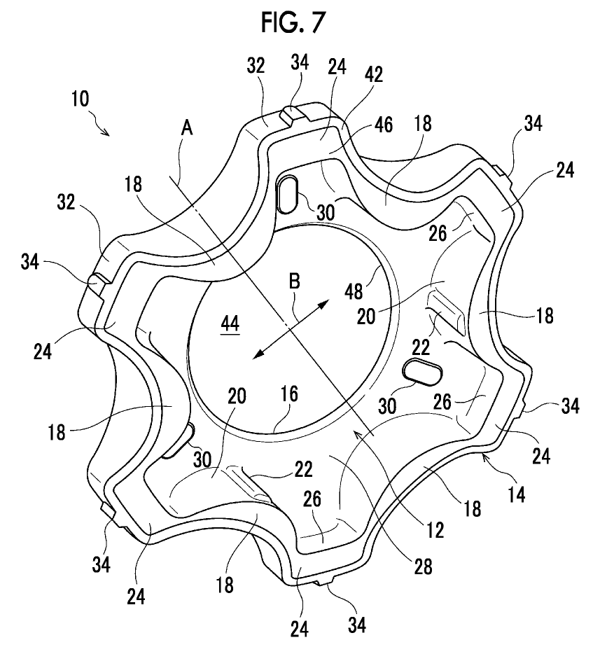

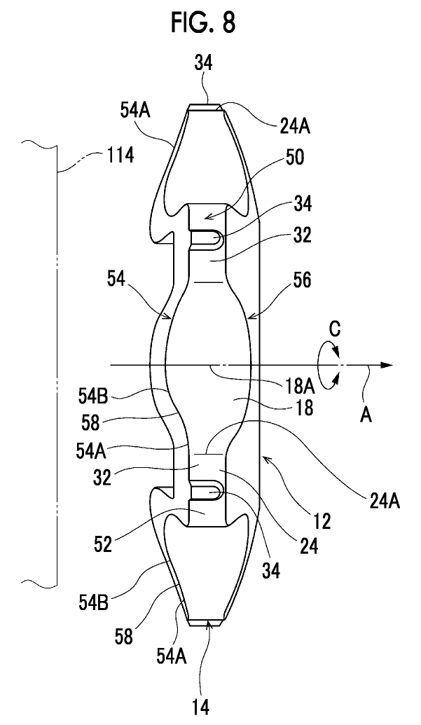

FIG. 4 is a front view of the attachment 10, and is a front view of the attachment 10 as viewed from one side (side opposite to a side on which the case 114 is disposed) in the rotation axis A direction. FIG. 5 is a rear view of the attachment 10, and is a rear view of the attachment 10 as viewed from the other side (side on which the case 114 is disposed) opposite to one side in the rotation axis A direction. FIGS. 6 and 7 are perspective views showing the attachment 10 shown in FIG. 5 in different directions. FIG. 8 is a side view of the attachment 10, and is a side view of the attachment 10 as viewed from the outer side in the radial direction B.

As shown in FIGS. 4-8, the attachment 10 includes a top surface portion 12 and a side wall portion 14. The top surface portion 12 is provided at a position facing the end surface 160 of the bending operation knob 118 in the rotation axis A direction. In addition, the top surface portion 12 includes an opening portion 16 at a central portion thereof.

In a case where the attachment 10 is attached to the bending operation knob 118, the top surface portion 12 covers the end surface 160 of the bending operation knob 118, and the lock dial 122 protrudes to the outside from the opening portion 16. The top surface portion 12 is an example of a top surface portion according to the present invention.

The side wall portion 14 is provided to extend from an edge portion of the top surface portion 12 toward the other side (side on which the case 114 is disposed) in the rotation axis A direction. In a case where the attachment 10 is attached to the bending operation knob 118, the side wall portion 14 is configured to cover the outer peripheral portion 152 of the bending operation knob 118. The side wall portion 14 is an example of a side wall portion according to the present invention.

The side wall portion 14 includes a first form portion 18. The first form portion 18 has a shape corresponding to the recessed surface portion 154 (see FIG. 2) of the bending operation knob 118, and is provided at a position facing the recessed surface portion 154 in the radial direction B. The first form portion 18 includes a facing surface 20 that faces the recessed surface portion 154 in the radial direction B. In addition, the first form portion 18 includes a first protruding portion 22 that is provided on the facing surface 20 and protrudes toward the recessed surface portion 154.

The first protruding portion 22 is provided at a central portion (portion located on the inner side in the radial direction B) of the facing surface 20 such that the facing surface 20 and the recessed surface portion 154 do not directly come into contact with each other in a case where the attachment 10 is attached to the bending operation knob 118. That is, the first protruding portion 22 is provided at a top of an arc portion of the facing surface 20 that is arc-shaped and is curved toward the inner side in the radial direction B. As a result, in a case where the attachment 10 is attached to the bending operation knob 118, the first protruding portion 22 comes into contact with the recessed surface portion 154, and the facing surface 20 is disposed with a gap with respect to the recessed surface portion 154. The first protruding portion 22 is formed of an elastic member such that the first protruding portion 22 is elastically deformed in a case of coming into contact with the recessed surface portion 154 as described below. The first form portion 18, the facing surface 20, and the first protruding portion 22 are examples of a first form portion, a first facing surface, and a first protruding portion according to the present invention, respectively.

Here, in the attachment 10 according to the embodiment, the first protruding portion 22 is provided at a position corresponding to three recessed surface portions 154 that are every other one of the six recessed surface portions 154 along the circumferential direction C. That is, the three first protruding portions 22 are configured to be disposed at equal intervals (including a case of substantially equal intervals) in the circumferential direction C. As a result, in a case where the attachment 10 is attached to the bending operation knob 118, the three first protruding portions 22 can come into contact with the three corresponding recessed surface portions 154 from three different directions.

Further, in the attachment 10 according to the embodiment, the first protruding portion 22 is provided to extend in the rotation axis A direction and has an elongated shape having the rotation axis A direction as a longitudinal direction. As a result, in a case where the attachment 10 is attached to the bending operation knob 118, the three first protruding portions 22 can be in surface contact with the three corresponding recessed surface portions 154.

The side wall portion 14 includes a second form portion 24. The second form portion 24 has a shape corresponding to the protruding surface portion 156 (see FIG. 2) and is provided at a position facing the protruding surface portion 156 in the radial direction B. The second form portion 24 includes a facing surface 26 that faces the protruding surface portion 156 in the radial direction B. The second form portion 24 is an example of a second form portion according to the present invention.

The top surface portion 12 includes a facing surface 28 that faces the end surface 160 (see FIG. 2) of the bending operation knob 118 in the rotation axis A direction. The facing surface 28 includes a second protruding portion 30 that protrudes toward the end surface 160. The second protruding portion 30 is disposed in a region of the facing surface 28 on an inner side of the second form portion 24 in the radial direction B.

In a case where the attachment 10 is attached to the bending operation knob 118, the second protruding portion 30 comes into contact with the end surface 160 in the rotation axis A direction. The facing surface 28 and the second protruding portion 30 are examples of a second facing surface and a second protruding portion according to the present invention, respectively.

The second protruding portion 30 is provided at a position corresponding to three protruding surface portions 156 that are every other one of the six protruding surface portions 156 (see FIG. 2) along the circumferential direction C. As a result, in a case where the attachment 10 is attached to the bending operation knob 118, the three second protruding portions 30 that are disposed at equal intervals (including substantially equal intervals) in the circumferential direction C can come into contact with the end surface 160.

In addition, the second protruding portion 30 is provided to extend in the radial direction B and has an elongated shape (elliptical shape) having the radial direction B as a longitudinal direction. As a result, in a case where the attachment 10 is attached to the bending operation knob 118, the three second protruding portions 30 can be in surface contact with the end surface 160.

A protrusion 34 is provided on an outer side surface 32 of the second form portion 24 that faces the outer side in the radial direction B in order to easily hook a finger of the operator during the bending operation of the endoscope 100. Since the outer side surface 32 of the second form portion 24 according to the embodiment is formed in a flat shape without unevenness, the protrusion 34 that is provided to protrude from the outer side surface 32 toward the outer side in the radial direction B makes it easier to hook the finger of the operator than in a configuration without the protrusion 34.

Here, in a case where the operator grips the operating portion 104, the bending operation knobs 116, 118 are provided on a side surface opposite to the side surface of the operating portion 104 on which the palm is in contact. That is, the bending operation knobs 116, 118 on the side opposite to the palm side need to be rotationally operated by the finger of the hand that grips the operating portion 104, and the operator having a relatively small hand may have difficulty in reaching the bending operation knobs 116, 118 with the finger and operating the bending operation knobs 116, 118. In particular, the bending operation knob 118 for left-right operation has a smaller diameter than the bending operation knob 116 for up-down operation and is located at a position farther from the operating portion 104 than the bending operation knob 116, and thus may be difficult to operate. Therefore, since the attachment 10 is attached to the bending operation knob 118 and the protrusion 34 is provided on the outer side surface 32 of the second form portion 24, even an operator having a small hand can easily reach the protrusion 34 with the finger, and the rotation operation of the bending operation knob 118 can be easily performed. The protrusion 34 configured as described above is provided on each of the outer side surfaces 32 of the six second form portions 24. The protrusion 34 is an example of a protrusion according to the present invention.

Next, a method of attaching the attachment 10 to the bending operation knob 118 will be described. The attachment 10 is attached to the bending operation knob 118 by being pushed from the outer side of the bending operation knob 118 toward the bending operation knob 118. Hereinafter, the description thereof will be made in detail.

First, each first form portion 18 of the attachment 10 is aligned with each recessed surface portion 154 of the bending operation knob 118 in the rotation axis A direction. In a case where the attachment 10 is pushed from the outer side of the bending operation knob 118 toward the bending operation knob 118 in this state, first, the three first protruding portions 22 of the attachment 10 come into contact with the three recessed surface portions 154 of the bending operation knob 118 corresponding to the three first protruding portions 22, respectively.

Thereafter, in a case where the attachment 10 is further pushed in the same direction, the three first protruding portions 22 are elastically deformed toward the outer side in the radial direction B by a reaction force received from the three recessed surface portions 154 and come into surface contact with the three recessed surface portions 154, respectively. In a case of being pushed in, the attachment 10 is pushed against the bending operation knob 118 without wobbling because the three first protruding portions 22 come into contact with the three corresponding recessed surface portions 154 from three different directions. Further, since the three first protruding portions 22 of the attachment 10 have an elongated shape having the rotation axis A direction as a longitudinal direction, the attachment 10 is pushed against the bending operation knob 118 in a stable posture.

In a case where the attachment 10 is pushed to a position where the attachment 10 is completed to be attached to the bending operation knob 118, a substantially entire length portion of the three first protruding portions 22 in the rotation axis A direction is elastically in close contact with the three recessed surface portions 154, and the three second protruding portions 30 are in close contact with the end surface 160. As a result, the attachment 10 is attached to the bending operation knob 118.

The attachment 10 attached to the bending operation knob 118 is attached to the bending operation knob 118 in a stable posture without wobbling because the three first protruding portions 22 are in surface contact with the three recessed surface portions 154, respectively. In addition, since the attachment 10 is attached to the bending operation knob 118 by a so-called three-point support structure in which the three second protruding portions 30 that are disposed at equal intervals in the circumferential direction C are in surface contact with the end surface 160, the attachment 10 is attached to the bending operation knob 118 in a more stable posture.

As described above, according to the attachment 10 of the embodiment, the attachment 10 can be attached to the bending operation knob 118 only by bringing the three first protruding portions 22 provided in the attachment 10 into close contact with the three recessed surface portions 154 provided in the bending operation knob 118. In addition, in a case where the attachment 10 is pulled in a direction opposite to the pushing direction, the three first protruding portions 22 are detached from the three recessed surface portions 154, so that the attachment 10 can be easily removed from the bending operation knob 118.

Therefore, according to the attachment 10 of the embodiment, since a configuration is adopted in which the first protruding portion 22 that protrudes toward the recessed surface portion 154 is provided on the facing surface 20 of the first form portion 18 in the side wall portion 14, and the first protruding portion 22 is brought into contact with the recessed surface portion 154 as the attachment structure of the attachment 10 with respect to the bending operation knob 118, the attachment 10 is easily attachable to and detachable from the bending operation knob 118 of the endoscope 100.

In addition, according to the attachment 10 of the embodiment, since the first protruding portion 22 is formed along the longitudinal direction of the support shaft 130 (rotation axis A) and the attachment 10 is inserted and pulled out along this direction, the attachment 10 is further easily attachable to and detachable from the bending operation knob 118.

In addition, according to the attachment 10 of the embodiment, the three first protruding portions 22 can be attached to the bending operation knob 118 in a state of coming into contact with and being in surface contact with the three corresponding recessed surface portions 154 from different directions. Therefore, the attachment 10 can be stably attached to the bending operation knob 118 without wobbling.

In the embodiment, one of the preferred aspects is described in which the three first protruding portions 22 are provided in the attachment 10, but the present invention is not limited thereto. For example, the configuration may be such that more first protruding portions 22 than those in the embodiment are provided as in the modification example described below, or the configuration may be such that less first protruding portions 22 than those in the embodiment are provided. That is, the configuration may be such that at least one first protruding portion 22 is provided.

In addition, in the embodiment, one of the preferred aspects is described in which the first protruding portion 22 has an elongated shape having the rotation axis A direction as a longitudinal direction, but the first protruding portion 22 may have another shape (for example, a circular shape or a polygonal shape) as long as the first protruding portion 22 can be in surface contact with the recessed surface portion 154. In addition, the first protruding portion 22 is not limited to the configuration of being in surface contact with the recessed surface portion 154, and may be in point contact with the recessed surface portion 154.

In addition, according to the attachment 10 of the embodiment, in addition to the configuration including the three first protruding portions 22 described above, the configuration in which the three second protruding portions 30 that are disposed at equal intervals (including substantially equal intervals) in the circumferential direction C are in surface contact with the end surface 160 is provided, so that the attachment of the attachment 10 to the bending operation knob 118 can be further stabilized.

In the embodiment, one of the preferred aspects is described in which the three second protruding portions 30 are provided in the attachment 10, but the present invention is not limited thereto. For example, the configuration may be such that more second protruding portions 30 than those in the embodiment are provided as in the modification example described below.

In addition, in the attachment 10 of the embodiment, since the protrusion 34 is provided on the outer side surface 32 of the second form portion 24 that faces the outer side in the radial direction B, during the bending operation of the endoscope 100, the rotation operation of the attachment 10 can be easily performed by hooking the finger of the operator on the protrusion 34.

### Other characteristic configurations

Hereinafter, other characteristic configurations of the attachment 10 of the embodiment will be described.

The attachment 10 has an appearance with a color different from the bending operation knob 118. For example, in a case where the color of the appearance of the bending operation knob 118 is black, the color of the appearance of the attachment 10 is light gray. According to such a configuration, it is possible to easily recognize whether or not the attachment 10 is attached to the bending operation knob 118 by simply looking at the endoscope 100 from the outside.

In addition, the attachment 10 is configured as a structural body 40 including the top surface portion 12 and the side wall portion 14. The structural body 40 is configured as a double-layer structure including an exterior member 42 and an interior member 46. The structural body 40 is a two-color molded product in which the exterior member 42 and the interior member 46 are integrated. The structural body 40, the exterior member 42, and the interior member 46 are examples of a structural body, an exterior member, and an interior member according to the present invention, respectively.

The outer side of the interior member 46 is surrounded by the exterior member 42. In addition, a space portion 44 that accommodates the bending operation knob 118 is defined by the inner side of the interior member 46. According to the structural body 40 of the present example, six protrusions 34 are formed in the exterior member 42, and three first protruding portions 22 and three second protruding portions 30 are formed in the interior member 46, respectively.

In a case where an example of a material constituting the exterior member 42 and the interior member 46 is described, the exterior member 42 is made of polycarbonate (PC), and the interior member 46 is made of an elastomer (for example, polyester-based elastomer).

In a case where the attachment 10 is attached to the bending operation knob 118, the three first protruding portions 22 made of an elastomer and having elasticity are in close contact with the three recessed surface portions 154, respectively, and the three second protruding portions 30 are in close contact with the end surface 160. As a result, the attachment 10 can be reliably attached to the bending operation knob 118, and the attachment 10 can be prevented from unintentionally falling off from the bending operation knob 118. In addition, since the exterior member 42 is made of hard PC, a direct operation feeling that the operator who rotationally operates the attachment 10 feels as if directly operating the bending operation knob 118 can be given.

In the embodiment, the structural body 40 including the exterior member 42 made of PC and the interior member 46 made of an elastomer is illustrated, but the present invention is not limited thereto. The interior member 46 may be made of a material having a lower hardness than the exterior member 42. In addition, from the viewpoint of the attachability of the attachment 10 to the bending operation knob 118, the interior member 46 is preferably an elastic member. As a result, in a case where the attachment 10 is attached to the bending operation knob 118, the interior member 46 is slightly deformed to fit the bending operation knob 118, so that the attachability is improved. In addition, from the viewpoint of direct operability, the exterior member 42 is preferably a rigid member. As a result, since the exterior member 42 is not deformed in a case of operating the bending operation knob 118, a direct operation feeling can be given to the operator. Here, the hardness refers to, for example, hardness obtained by a Rockwell hardness test or a durometer hardness test. The interior member 46 is not limited to being made of an elastomer and may be made of rubber.

In addition, the structural body 40 includes the opening portion 16 that penetrates the top surface portion 12, and an edge portion 48 of the opening portion 16 is made of the interior member 46. That is, the exterior member 42 and the interior member 46 are each provided with an opening portion (not shown) for defining the opening portion 16, and the edge portion 48 is made of the interior member 46 made of rubber by covering the edge portion of the opening portion of the exterior member 42 with the edge portion of the opening portion of the interior member 46. The advantages of making the edge portion 48 of rubber will be described below.

In a case where the attachment 10 is attached to the bending operation knob 118 in a state where the edge portion 48 of the attachment 10 is in contact with the outer peripheral portion of the lock dial 122, the edge portion 48 may be rubbed against the outer peripheral portion of the lock dial 122 and may be scratched in a case where the attachment 10 is rotationally operated. Since the edge portion 48 of the attachment 10 of the embodiment is made of rubber, the edge portion 48 can be prevented from being scratched.

The structural body 40 of the embodiment is configured as a double-layer structure including the exterior member 42 and the interior member 46, but the present invention is not limited to this configuration. For example, the structural body 40 may be configured as a triple structure in which an intermediate member is interposed between the exterior member 42 and the interior member 46. That is, the structural body 40 may be configured as a double-layer structure including at least the exterior member 42 and the interior member 46.

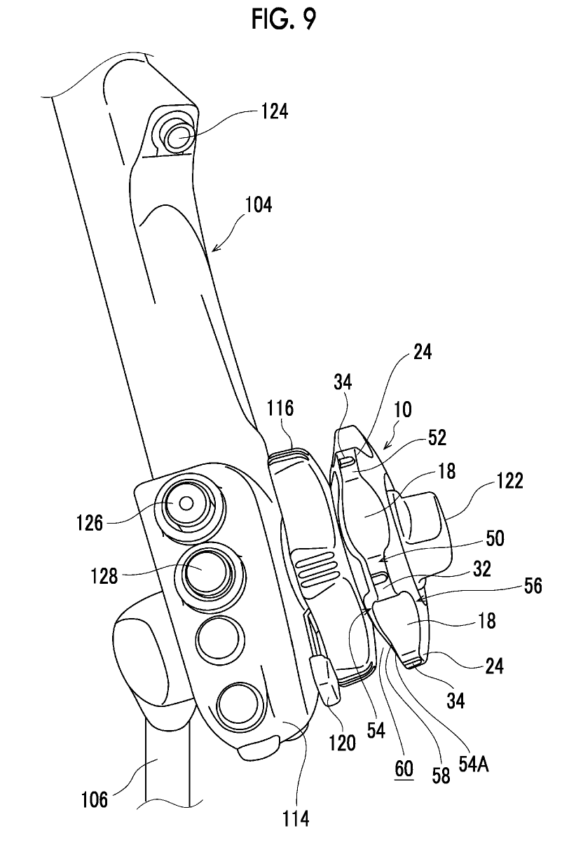

FIG. 9 is an enlarged perspective view showing a state in which the attachment 10 is attached to the bending operation knob 118. In FIG. 9, since the bending operation knob 118 is covered by the attachment 10, the bending operation knob 118 is not shown, and a state in which the attachment 10 is disposed to approach the bending operation knob 116 in the rotation axis A direction is shown.

As shown in FIGS. 8 and 9, an outer peripheral portion 50 of the attachment 10 includes an outer peripheral surface 52 (including the outer side surface 32) that faces the outer side in the radial direction B of the outer peripheral portion 50 and two ridge portions 54, 56 that are interposed between the outer peripheral surfaces 52 in the rotation axis A direction. The ridge portion 54 is located on a side facing the bending operation knob 116, and the ridge portion 56 is located on a side opposite to the ridge portion 54 across the outer peripheral surface 52.

The two ridge portions 54, 56 are each sharp. In addition, among the two ridge portions 54, 56, the two ridge portions 54, 56 located at the second form portion 24 of the attachment 10 are sharper than the two ridge portions 54, 56 located at the first form portion 18 of the attachment 10. As a result, the finger of the operator can be securely hooked on the second form portion 24.

In addition, the two ridge portions 54, 56 are curved to approach each other from a bottom portion 18A of the first form portion 18 toward an end portion 24A of the second form portion 24 in the rotation axis A direction.

In addition, the ridge portion 54 located at the first form portion 18 has an inflection portion 58 at a substantially central position from the bottom portion 18A of the first form portion 18 toward the end portion 24A of the second form portion 24. Then, a ridge portion 54B from the bottom portion 18A toward the inflection portion 58 of the ridge portion 54 is curved in a direction of bulging toward the bending operation knob 116, and a ridge portion 54A from the inflection portion 58 toward the end portion 24A of the second form portion 24 is curved in a direction of bulging toward the ridge portion 56. That is, in a case where the ridge portion 54A is viewed from the outer side in the radial direction B, the ridge portion 54A is curved in a direction away from the bending operation knob 116 in the rotation axis A direction.

According to the attachment 10 of the embodiment, since the inflection portion 58 is provided on the ridge portion 54 on the side facing the bending operation knob 116 among the ridge portions 54, 56 of the outer peripheral portion 50, and the ridge portion 54A from the inflection portion 58 toward the end portion 24A of the second form portion 24 of the ridge portion 54 is curved in a direction of bulging toward the ridge portion 56, a gap 60 can be formed between the bending operation knob 116 and the attachment 10 in the rotation axis A direction. By using the gap 60 as a gap for removing the finger of the operator, the operator can smoothly perform the finger removing operation.

In the embodiment, the attachment 10 that is attachable to and detachable from the bending operation knob 118 of the endoscope 100 has been described, but the present invention is not limited thereto. For example, the attachment for a medical instrument according to the present invention can be applied to an operating member of an ultrasound probe comprising an insertion portion that is inserted into a body.

Hereinafter, several modification examples will be described with reference to the drawings.

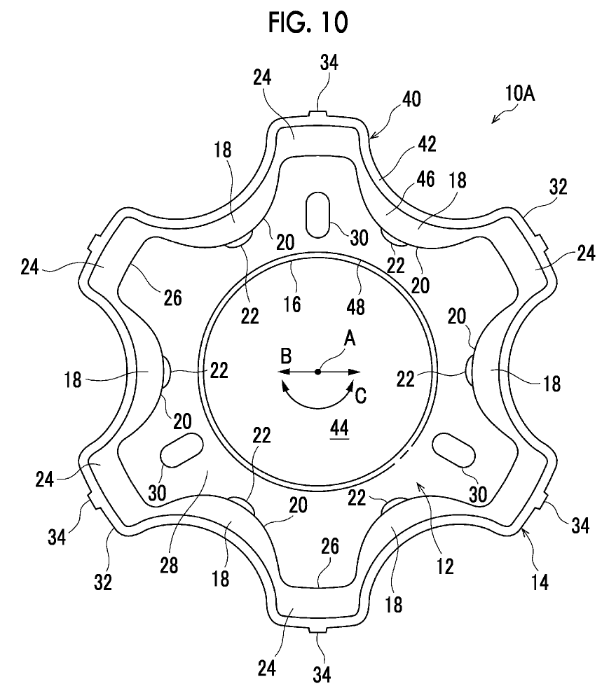

FIG. 10 is a rear view of an attachment 10A that is a first modification example. In a case of describing the attachment 10A, the same or similar members as the attachment 10 shown in FIG. 5 will be designated by the same reference numerals and will not be described.

A difference between the attachment 10A shown in FIG. 10 and the attachment 10 shown in FIG. 5 is that the attachment 10 of FIG. 5 is configured such that the first protruding portion 22 is provided in three first form portions 18 that are every other one of the six first form portions 18 along the circumferential direction C, whereas the attachment 10A shown in FIG. 10 is configured such that the first protruding portion 22 is provided in all of the six first form portions 18.

According to the attachment 10A shown in FIG. 10, in a case where the attachment 10A is attached to the bending operation knob 118, the six first protruding portions 22 are in surface contact with the six recessed surface portions 154 of the bending operation knob 118, respectively, and the attachment 10A is attached to the bending operation knob 118. As a result, the attachment 10A is stably attached to the bending operation knob 118 without wobbling.

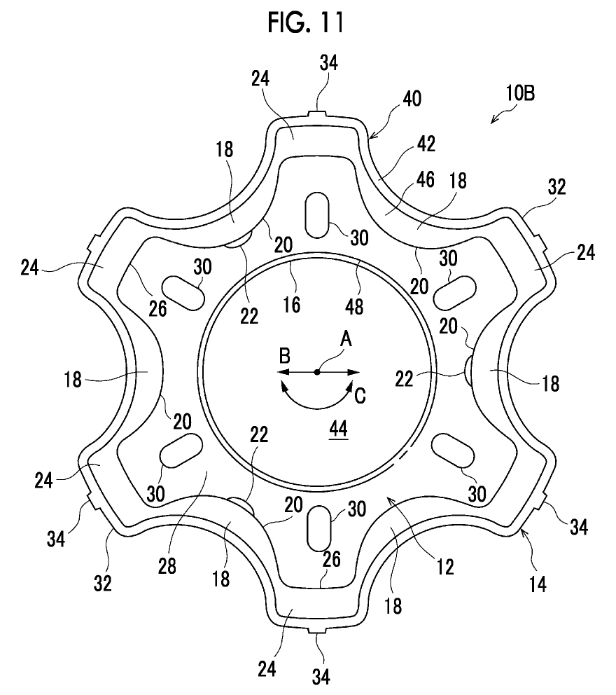

FIG. 11 is a rear view of an attachment 10B that is a second modification example. In a case of describing the attachment 10B, the same or similar members as the attachment 10 shown in FIG. 5 will be designated by the same reference numerals and will not be described.

A difference between the attachment 10B shown in FIG. 11 and the attachment 10 shown in FIG. 5 is that the attachment 10 of FIG. 5 is configured such that the second protruding portion 30 is provided at a position corresponding to three second form portions 24 that are every other one of the six second form portions 24 along the circumferential direction C, whereas the attachment 10B shown in FIG. 11 is configured such that the second protruding portion 30 is provided at a position corresponding to all of the six second form portions 24.

According to the attachment 10B shown in FIG. 11, in a case where the attachment 10B is attached to the bending operation knob 118, the six second protruding portions 30 are in surface contact with the end surface 160 of the bending operation knob 118, respectively, and the attachment 10B is attached to the bending operation knob 118. As a result, the attachment 10B is stably attached to the bending operation knob 118 without wobbling.

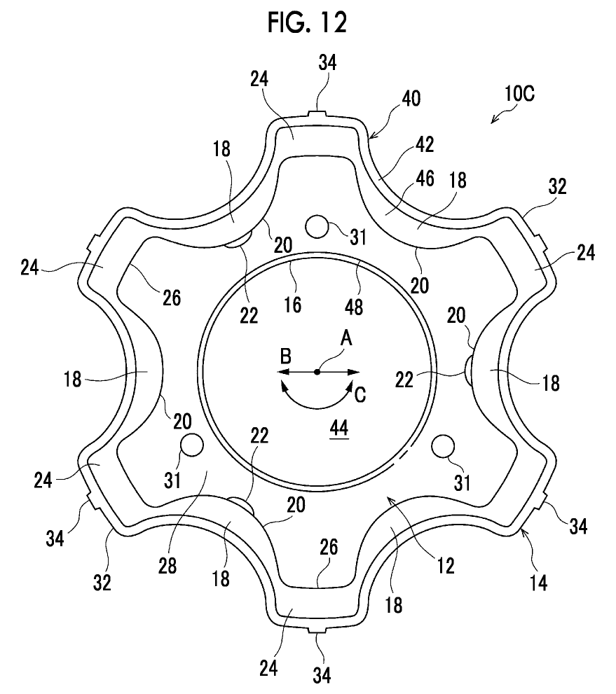

FIG. 12 is a rear view of an attachment 10C that is a third modification example. In a case of describing the attachment 10C, the same or similar members as the attachment 10 shown in FIG. 5 will be designated by the same reference numerals and will not be described.

A difference between the attachment 10C shown in FIG. 12 and the attachment 10 shown in FIG. 5 is that the second protruding portion 30 of the attachment 10 of FIG. 5 is formed in an elongated shape having the radial direction B as a longitudinal direction, whereas a protruding portion 31 (second protruding portion according to the present invention) of the attachment 10C shown in FIG. 12 is formed in a hemispherical shape.

According to the attachment 10C shown in FIG. 12, in a case where the attachment 10C is attached to the bending operation knob 118, three hemispherical protruding portions 31 come into contact with the end surface 160 of the bending operation knob 118, respectively, and the attachment 10C is attached to the bending operation knob 118. As a result, since the attachment 10C is supported by the bending operation knob 118 by so-called three-point contact, the attachment 10C is stably attached to the bending operation knob 118 without wobbling.

Although the embodiment of the attachment for a medical instrument according to the present invention has been described above, the present invention may be improved or modified in various ways without departing from the spirit of the present invention.

### EXPLANATION OF REFERENCES

10: bending operation knob attachment
10A: attachment
10B: attachment
10C: attachment
12: top surface portion
14: side wall portion
16: opening portion
18: first form portion
18A: bottom portion
20: facing surface
22: first protruding portion
24: second form portion
24A: end portion
26: facing surface
30: second protruding portion
32: outer side surface
34: protrusion
40: structural body
42: exterior member
44: space portion
46: interior member
50: outer peripheral portion
52: outer peripheral surface
54: ridge portion
54A: ridge portion
54B: ridge portion
56: ridge portion
58: inflection portion
60: gap
100: endoscope
102: insertion portion
104: operating portion
106: universal cord
108: distal end portion
110: bendable portion
112: soft portion
114: case
115: opening portion
116: bending operation knob
118: bending operation knob
120: lock lever
122: lock dial
124: forceps port
126: air/water supply button
128: suction button
130: support shaft
132: rotation drive cylinder
134: rotation drive cylinder
138: sprocket
140: chain
142: sprocket
144: chain
146: locking mechanism
148: locking mechanism
150: opening portion
152: outer peripheral portion
154: recessed surface portion
156: protruding surface portion
158: wall portion
160: end surface
A: rotation axis
B: radial direction
C: circumferential direction

What is claimed is:

1. An attachment for a medical instrument, the attachment being attachable to and detachable from an operating member rotatably provided in an operating portion of the medical instrument, in which a recessed surface portion for hooking a finger, which is recessed inward in a radial direction orthogonal to a rotation axis direction of the operating member, is provided in an outer peripheral portion of the operating member, the attachment comprising:
a top surface portion provided at a position facing an end surface of the operating member on one side in the rotation axis direction; and
a side wall portion extending from an edge portion of the top surface portion toward the other side opposite to the one side in the rotation axis direction,
wherein the side wall portion includes a first form portion provided at a position facing the recessed surface portion, and
the first form portion includes a first protruding portion provided on a first facing surface that faces the recessed surface portion and protruding toward the recessed surface portion.

2. The attachment for a medical instrument according to claim 1,
wherein a plurality of the recessed surface portions are provided in the outer peripheral portion of the operating member, and
the first protruding portion is provided at a position corresponding to only a part of the plurality of recessed surface portions.

3. The attachment for a medical instrument according to claim 1,
wherein a plurality of the recessed surface portions are provided in the outer peripheral portion of the operating member along a circumferential direction centered on the rotation axis direction, and
the first protruding portion is provided at a position corresponding to every other recessed surface portion among the plurality of recessed surface portions along the circumferential direction.

4. The attachment for a medical instrument according to claim 1,
wherein a protruding surface portion disposed on an outer side of the recessed surface portion in the radial direction is provided in the outer peripheral portion of the operating member,
the side wall portion includes a second form portion provided at a position facing the protruding surface portion, and
the top surface portion includes a second protruding portion disposed in a region of a second facing surface that faces the end surface on an inner side of the second form portion in the radial direction and protruding toward the end surface.

5. The attachment for a medical instrument according to claim 4,
wherein a plurality of the recessed surface portions and a plurality of the protruding surface portions are alternately provided in the outer peripheral portion of the operating member along a circumferential direction centered on the rotation axis direction,
the first protruding portion is provided at a position corresponding to every other recessed surface portion among the plurality of recessed surface portions along the circumferential direction, and
the second protruding portion is provided at a position corresponding to every other protruding surface portion among the plurality of protruding surface portions along the circumferential direction.

6. The attachment for a medical instrument according to claim 1,
wherein the first protruding portion is provided to extend in the rotation axis direction.

7. The attachment for a medical instrument according to claim 4,
wherein the second form portion includes a protrusion provided on an outer side surface facing outward in the radial direction.

8. The attachment for a medical instrument according to claim 1,
wherein the attachment for a medical instrument has an appearance with a color different from a color of the operating member.

9. The attachment for a medical instrument according to claim 1,
wherein a structural body including the top surface portion and the side wall portion is configured as at least a double-layer structure including an exterior member and an interior member surrounded by the exterior member and defining a space portion that accommodates the operating member.

10. The attachment for a medical instrument according to claim 9,
wherein the interior member is made of a material having a lower hardness than a hardness of the exterior member.

11. The attachment for a medical instrument according to claim 10,
wherein the exterior member is a rigid member, and
the interior member is an elastic member.

12. The attachment for a medical instrument according to claim 10,
wherein the structural body is a two-color molded product in which the exterior member and the interior member are integrated.

13. The attachment for a medical instrument according to claim 9,
wherein the structural body includes an opening portion penetrating the top surface portion, and
an edge portion of the opening portion is made of the interior member.

14. The attachment for a medical instrument according to claim 1,
wherein the medical instrument is an endoscope, and
the operating member is a bending operation member receiving a bending operation on an insertion portion of the endoscope.

15. A medical instrument system comprising:
the medical instrument; and
the attachment for a medical instrument according to claim 1.

16. The medical instrument system according to claim 15,
wherein the medical instrument is an endoscope, and
the operating member is a bending operation member receiving a bending operation on an insertion portion of the endoscope.
